(19) United States
(12) Patent Application Publication
(10) Pub. No.: US 2019/0142507 A1
(43) Pub. Date: May 16, 2019

(54) FISTULA CREATION FOR FLUID DRAINAGE

(71) Applicant: Spiration Inc. (dba Olympus Respiratory America), Redmond, WA (US)

(72) Inventors: Brandon J. Shuman, Kirkland, WA (US); Sujeeth Parthiban, Bothell, WA (US)

(21) Appl. No.: 15/811,948

(22) Filed: Nov. 14, 2017

(51) Int. Cl. A61B 18/14; A61B 18/12; A61B 17/122

(52) U.S. Cl. CPC A61B 18/1492; A61B 18/1206; A61B 2018/00083; A61B 2018/126; A61B 17/1227

(57) ABSTRACT

A fistula creation system for quickly creating a fistula between a first biological tissue and a second biological tissue of a body. The system includes a tissue fastening tool for applying pressure to a periphery around an area of a first biological tissue and a second biological tissue of a body, a power source and a fistula creating device. The fistula creating device includes one or more electrodes located at the distal end of a catheter and are in electrical communication with the power source. The electrodes cut the first and second biological tissue within the area when charged by the power source. The fistula creating device includes a balloon with an electrode for expanding the size of the fistula.

### BACKGROUND

Surgical bypass (anastomosis) of the gastroenterological (GI), vascular, or urological tracts are typically formed by cutting holes in the tracts at two locations and joining the holes with sutures or staples. The procedure is invasive, and subjects a patient to surgical risks such as bleeding, infection, pain, and adverse reaction to anesthesia. Additionally, an anastomosis created with sutures or staples can be complicated by post-operative leaks and adhesions. Leaks may result in infection or sepsis. Additionally, while traditional anastomosis procedures can be completed with an endoscope, laparoscope, or robot, it can be time consuming to join the holes in the tissues.

As an alternative to sutures or staples, surgeons can use mechanical springs or compression coils. In one example, these devices penetrate duodenum and common bile duct walls and apply a compressive force to the walls. An anastomosis is created as the tissue walls within the coils becomes necrotic, thus causing the coil to fall into the duodenum to be expelled by the body. After the anastomosis is formed fluid is allowed drained. However, it takes time for this anastomosis to be created.

### SUMMARY

The present invention includes a system for quickly creating a fistula between a first biological tissue and a second biological tissue of a body. The system includes a tissue fastening tool for applying pressure to a periphery around an area of a first biological tissue and a second biological tissue of a body, a power source and a fistula creating device. The fistula creating device includes a handle portion configured to be used exterior to a body, a catheter having a proximal end coupled to the handle portion and a distal end and one or more electrodes located at the distal end of the catheter. The one or more electrodes are in electrical communication with the power source and are configured to cut the first and second biological tissue within the area when charged by the power source.

In one aspect of the invention, the one or more electrodes includes two electrodes configured to operate in a bipolar manner. A first electrode of the two electrodes is located on an exterior surface of the catheter. A second electrode of the two electrodes is slidably located within the catheter.

In another aspect of the invention, a first electrode of the two electrodes is located on a first side of a lumen of the catheter and a second electrode of the two electrodes is located on a second side of the catheter.

In still another aspect of the invention, a patient pad is in electrical communication with the power source and the one or more electrodes are powered by the power source in a monopolar manner in conjunction with the patient pad.

In yet another aspect of the invention, the system includes a balloon attached to the catheter proximal from the distal end of the catheter and one or more balloon electrodes attached to an exterior surface of the balloon. The one or more electrodes includes a tip electrode located at a distal end of the catheter. The catheter includes a lumen exposed at a distal end to an interior of the balloon and at a proximal end to the handle portion. The lumen is configured to receive at least one of a gas or a fluid from the handle portion and pass the received gas or fluid to the balloon in order to inflate the balloon.

In a further aspect of the invention, a method includes applying pressure to a periphery around an area of a first biological tissue and a second biological tissue of a body, applying power to one or more electrodes and cutting the first and second biological tissues while advancing the powered one or more electrodes to and through the area, thereby creating a fistula between the first and second biological tissues.

In yet further aspects of the invention, while inflating a balloon located proximal from the distal end of the catheter, activating one or more balloon electrodes attached to an exterior surface of the balloon. The one or more electrodes includes a tip electrode located at a distal end of the catheter, wherein applying power to the one or more electrodes includes applying power to the tip electrode in association with a patient pad located external to the body. Applying power to the tip electrode is performed before inflating the balloon. The tip electrode and the balloon operate independently from each other. The tip electrode is used during device insertion into tissue, while the balloon electrode is operated after the device had penetrated the tissue.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 shows a system formed in accordance with an embodiment of the present invention;

FIG. 2 shows a component of the system deployed within an organ of the body;

FIG. 3 shows an example tissue fastening device formed in accordance with the prior art;

FIG. 4 shows the tissue fastening device of FIG. 3 deployed to attach tissue walls within a body;

FIG. 5 shows a perspective view of the distal end of a fistula creation device formed in accordance with an embodiment of the present invention;

FIG. 6 shows a side view of the tissue fastening device of FIG. 3 in use with the fistula creation device of FIG. 5;

FIG. 7 shows a perspective view of the distal end of a fistula creation device formed in accordance with an embodiment of the present invention;

FIG. 8 shows a side view of the tissue fastening device of FIG. 3 in use with the fistula creation device of FIG. 7;

FIG. 9 shows a perspective view of the distal end of a fistula creation and expanding device formed in accordance with an embodiment of the present invention;

FIG. 10 shows a side view of the tissue fastening device of FIG. 3 in use with the fistula creation and expanding device of FIG. 9;

FIG. 11-1 shows a side view of the fistula creation and expanding device of FIG. 9 in an expanded configuration;

FIG. 11-2 shows a cross-sectional end view of the device of FIG. 11-1 in a first expanded configuration in contact with tissue; and

FIG. 11-3 shows a cross-sectional end view of the device of FIG. 11-1 in a second expanded configuration in contact with tissue.

### DETAILED DESCRIPTION

In one embodiment, a device creates a fistula within a tissue fastening device that integrally fixes a first biological tissue to a second biological tissue and causes the tissues to communicate with each other. Note that the first and second biological tissues are not limited to different organs. For example, a region of an organ may be referred to as the first biological tissue and a different region of the same organ may be referred to as the second biological tissue, so as to include fixing these two regions within the same organ. In one embodiment, a treatment to drain a blocked common biliary duct by attaching it to a duodenum thus causing both the organs to communicate with each other will be described as an example.

An exemplary anastomosis procedure involves placing a tissue fastening device at targeted tissues, thus causing the tissues to come together. The tissue fastening device is deployed so that that a first portion maintains contact with an interior wall of a first lumen (e.g., common bile duct) and a second portion maintains contact with an interior wall of a second lumen (e.g., duodenum) to bring the tissue walls together. A fistula creating device cuts a whole in the tissue within the tissue fastening device using RF energy. By creating a whole within the compressed tissue walls before necrosis occurs, bile drainage is affected quickly for providing acute relief.

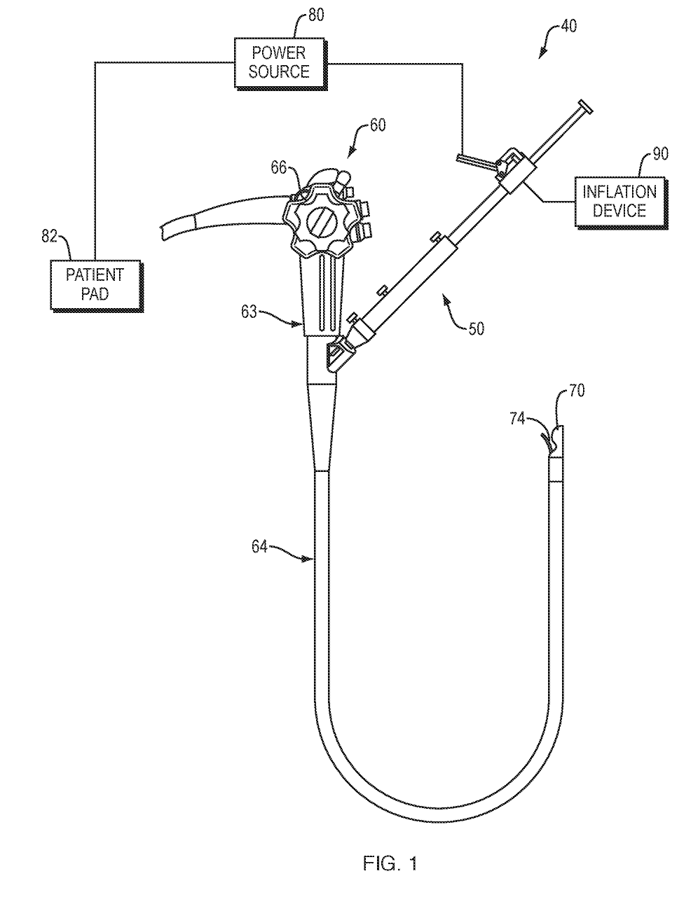

FIG. 1 shows a system 40 that includes a linear scanning type ultrasonic endoscope 60 (hereinafter simply referred to as an “endoscope”) as an example of an endoscope to be used together with a tissue fastening apparatus 50. The endoscope 60 is provided with a flexible insertion portion 64 that extends from a handle portion 63 to be used outside a body.

The handle portion 63 includes a knob 66 for controlling curvature of the distal end portion of the insertion portion 64. An ultrasonic transducer 70 is attached to the distal end of the insertion portion 64. The endoscope 60 may be provided with other probe-types of ultrasonic devices. An endoscope with a camera and light may be used without an ultrasound transducer. Additionally, an endoscope that is not provided with the ultrasonic transducer 70 can also be used. In this case, any imaging device used outside the body, such as an X-ray device, a magnetic resonance imaging (MM) device, or a computerized tomography (CT) device may be used jointly.

The distal end of the flexible insertion portion 64 includes a port 74 for delivery of a tissue fastening tool and/or a fistula creation tool (not shown) of the tissue fastening apparatus 50. The system 40 also includes a power source 80, a patient pad 82 and an inflation device 90. The power source 80 is in electrical communication with electrodes (not shown) located at a distal and of the fistula creation tool and the patient pad 82. The inflation device 90 is in pneumatic and or fluidic communication with a balloon device (not shown) located at a distal end of the fistula creation tool. The inflation device 90 may be any device capable of filling or draining a surgical balloons with a liquid and/or a gas.

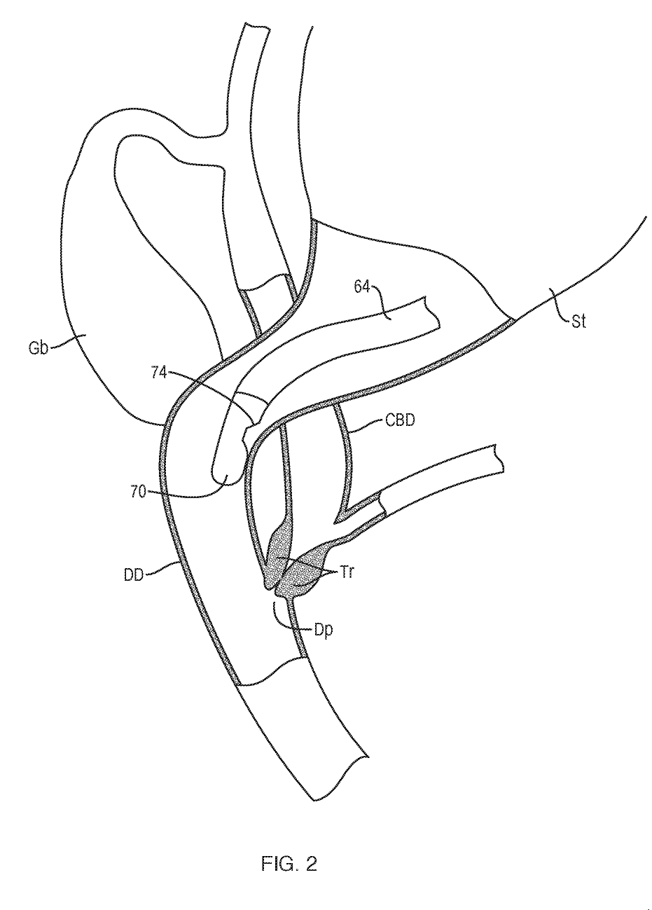

Next, the procedure of creating a fistula within an indwelled tissue fastening tool using the tissue fastening apparatus 50 configured as described above will be described. For example, as shown in FIG. 2, this kind of procedure is performed when a duodenal papilla (Dp) is obstructed, for example by a tumor (Tr), preventing bile drainage, and consequently the bile assimilates in the blood causing jaundice. This procedure enables the direct drainage of bile from the common biliary duct (CBD) to the duodenum (DD).

First, the flexible insertion portion 64 of the endoscope 60 is inserted into the patient's mouth then into the DD. The condition outside the DD is investigated by the ultrasonic transducer 70 or another imaging device, and an appropriate location for the procedure close to the CBD is targeted proximal from the Dp.

A tissue fastening tool (not shown) is deployed in stages from the port 74 to bond a wall of the DD to a wall of the CBD in order to create a permanent fistula (hole) between the CBD and the DD. Before the permanent fistula is created, a drainage fistula is created by activating a tissue fastening tool. The drainage fistula allows bile juice from the liver to drain into the DD. Permanent fistula creation is achieved by the tissue fastening tool compressing the CBD and DD membranes together causing stoppage of blood flow where the tissue is being compressed. The immediate result is interrupted blood flow where the tissue fastening tool is located and over time, membrane fusion around the tissue fastening tool and tissue necrosis where blood flow stopped. The necrotic tissue along with the tissue fastening tool will eventually fall into the duodenum, creating the permanent fistula through which the bile juice will drain.

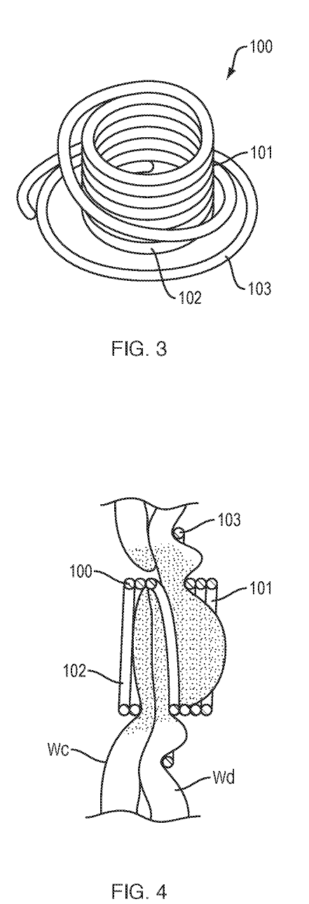

Hereinafter, one embodiment of the invention will be described. A tissue fastening apparatus in the present embodiment is an apparatus which integrally fixes a second biological tissue to a first biological tissue and causes the tissues to communicate with each other. As shown in FIG. 3, the apparatus includes a tissue fastening tool 100 that is deployed via the tissue fastening apparatus (FIG. 1).

As shown in FIGS. 3 and 4, the tissue fastening tool 100 includes a first tissue fixing section 101 which is hooked onto a wall of the duodenum Wd, a second tissue fixing section 102 which is hooked onto a wall of the common biliary duct Wc adjacent to the duodenum, and an outer peripheral spring portion 103 which is connected to the first tissue fixing section 101.

The tissue fastening tool 100 is formed of a highly elastic metal wire rod (hereinafter referred to as “metal wire rod”) in which all portions, i.e., the first tissue fixing section 101, the second tissue fixing section 102, and the outer peripheral spring portion 103 are wound into a coil shape. The first tissue fixing section 101 and the second tissue fixing section 102 have the same loop diameter, and are formed so that their mutual loops are coaxial with each other.

The spring portion 103 extends toward the second tissue fixing section 102 from the end of the first tissue fixing section 101 while forming a larger loop than the first tissue fixing section 101 and the second tissue fixing section 102. The loop that the spring portion 103 forms gradually becomes larger as it goes towards the second tissue fixing section 102. It is noted that this shape is not indispensable to the present invention, for example, the spring portion 103 may extend toward the second tissue fixing section 102, while forming a loop of different diameter(s).

Since the spring portion 103 extends towards the second tissue fixing section 102, as shown in FIGS. 3 and 4, the metal wire rod that forms the spring portion 103 has an angle so as to incline with respect to the axis of a loop (hereinafter referred to as a “basic loop”) of the first tissue fixing section 101 and the second tissue fixing section 102. The spring portion 103 may be formed so as to have one or more integer turns. The “one or more integer turns” means that an end of the spring portion 103 on the side of the first tissue fixing section 101 and an end of the spring portion 103 on the side of the end coil portion are aligned on the same straight line as the center without pinching the center of a basic loop therebetween in plan view of the tissue fastening tool 100.

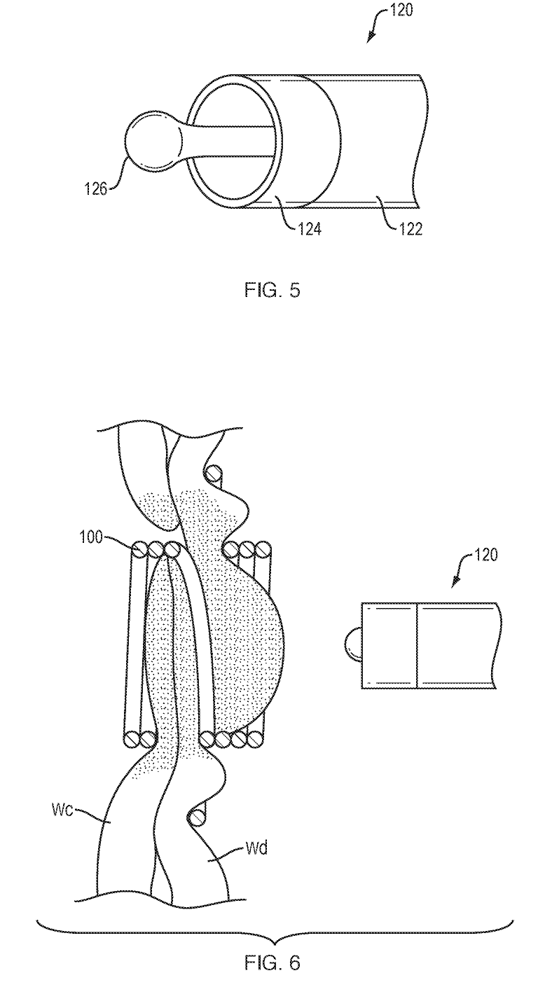

FIG. 5 shows a distal end of a fistula creating device 120. In one embodiment, the fistula creating device 120 includes a catheter 122 that includes a distal end and a proximal end. The distal end of the fistula creating device 120 includes a first electrode 124 at least partially formed circumferentially around the catheter 122. The catheter 122 also includes a lumen that receives a second electrode 126. The second electrode 126 may be slidably received within the lumen of the catheter 122. The catheter 122 may act as an insulator between the first and second electrodes 124 and 126 or an insulator material, such as a heat shrink, is applied over a portion of the second electrode 126 that may contact the catheter 122 at the location of the first electrode 124.

The first and second electrodes 124 and 126 are connected to the power source 80 via leads, traces and/or wires (not shown) and may operate in a bipolar cutting mode or may operate in conjunction with the patient pad 82 in a monopolar cutting mode. In one embodiment, only one of the electrodes 124 and 126 are included in the fistula creating device 120 for use in a monopolar cutting mode in conjunction with the patient pad 82. These cutting modes use radio frequency (RF) ablation to make a path through the tissue by vaporizing the tissue.

As shown in FIG. 6, at least one of the electrodes 124, 126 of the fistula creating device 120 are activated/charge/powered while or just before making contact with the tissue wall located within a central area of the deployed tissue fastening device 100. The heat generated by the first and second electrodes 124 and 126 causes cutting/ablation of the tissue wall. While electrode activation and tissue cutting are occurring, an operator advances the fistula creating device 120 through the deployed tissue fastening device 100 until of the tissue walls have been pierced. Once the fistula creating device 120 passes through both tissue walls compressed by the deployed tissue fastening device 100, the power supplied to the electrodes 124 and/or 126 is turned off. The deactivated fistula creating device 120 is then retracted from the deployed tissue fastening device 100, thus leaving a fistula for allowing fluid to pass between the tissue walls.

The fistula creating device 120 can be guided and deployed from the endoscope 60, such as that shown in FIG. 1. The proximal end of the fistula creating device 120 can include a handle that is received by the handle portion 63 of the endoscope 60, for example.

FIG. 7 shows a distal end of a fistula creating device 140 that includes a distal end and a proximal end. The fistula creating device 140 includes a catheter 142 that includes a first electrode 144 located at one side of a lumen of the catheter 142 and a second electrode 146 located at a second side of the lumen of the catheter 142. The lumen of the catheter 142 may include an insulator between the first and second electrodes 144 and 146 or insulator materials, such as heat shrink, is applied over at least a portion of the first and/or second electrodes 144, 146.

The first and second electrodes 144, 146 may be slidably received within the lumen of the catheter 142. The first and second electrodes 144 and 146 may be connected to the power source 80 such that they operate simultaneously in a bipolar manner mode for individually with the patient pad 82 in a monopolar mode. The proximal end of the fistula creating device 140 can include a handle that is received by the handle portion 63 of the endoscope 60, for example.

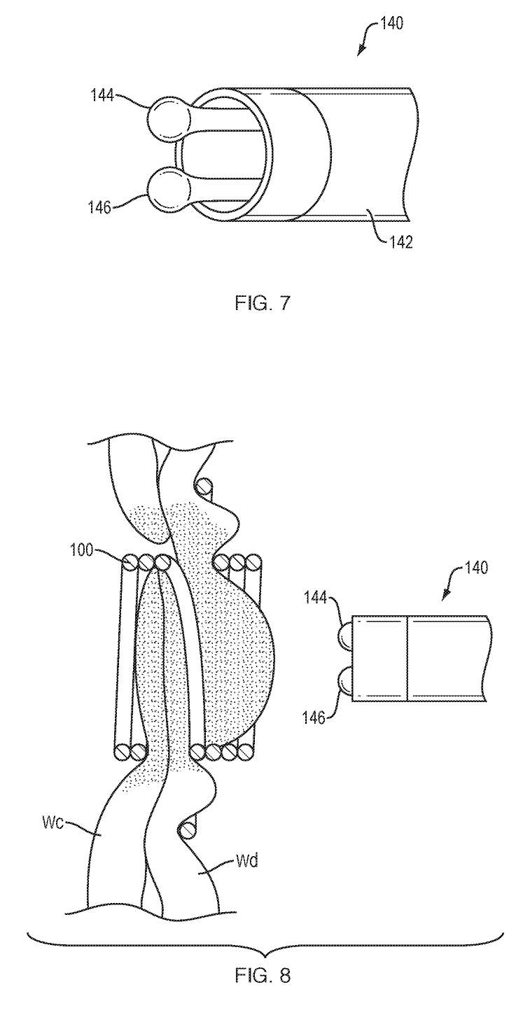

As shown in FIG. 8, the first and/or second electrodes 144, 146 of the fistula creating device 140 are activated/charged/powered upon while or just before making contact with the tissue wall located within a central area of the deployed tissue fastening device 100. The heat generated by the first and second electrodes 144 and 146 causes cutting/ablation of the tissue wall. While electrode activation and tissue cutting are occurring, an operator advances the fistula creating device 140 through the deployed tissue fastening device 100 until of the tissue walls have been pierced. Once the fistula creating device 140 passes through both tissue walls compressed by the deployed tissue fastening device 100, the power supplied to the electrodes 144 and/or 146 is turned off. The deactivated fistula creating device 140 is then retracted from the deployed tissue fastening device 100, thus leaving a fistula for allowing fluid to pass between the tissue walls.

The fistula creating device 140 to be guided and deployed from the endoscope 60 such as that shown in FIG. 1. The proximal end of the fistula creating device 140 can include a handle that is received by the handle portion 63 of the endoscope 60, for example.

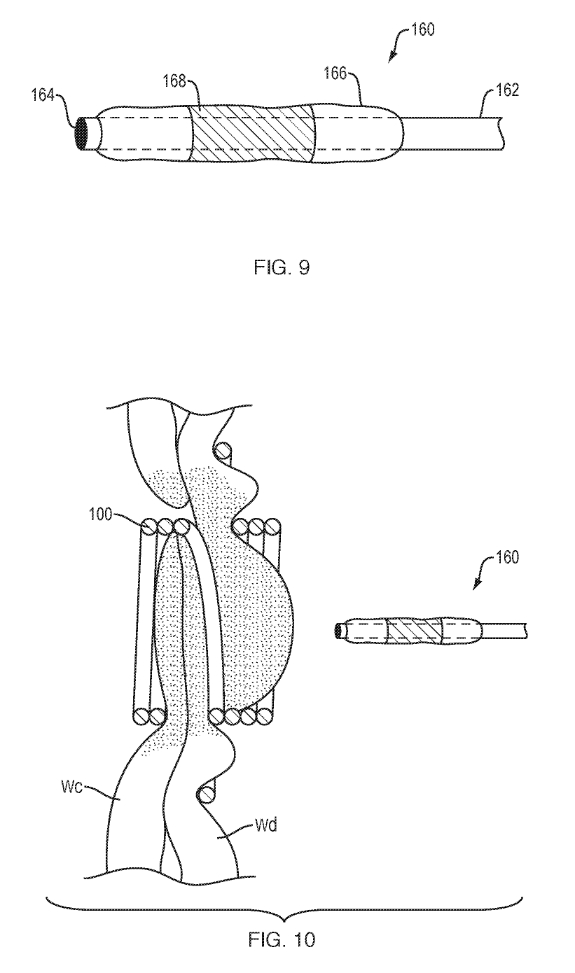

FIG. 9 shows a balloon fistula creating and expanding device 160 that includes a shaft 162 that passes through a scope device, such as that shown in FIG. 1. A proximal end of the shaft 162 includes a handle that includes features for connecting to the inflation device 90 and/or the power source 80. The handle is received by the handle portion 63 of the endoscope 60, for example. A distal end of the shaft 162 includes a distal tip electrode 164, a balloon 166 located proximal from the distal tip 164 and one or more electrodes 168 attached to a surface of the balloon 166. The electrodes 164, 168 are connected to the power source 80 via leads, traces and/or wires (not shown).

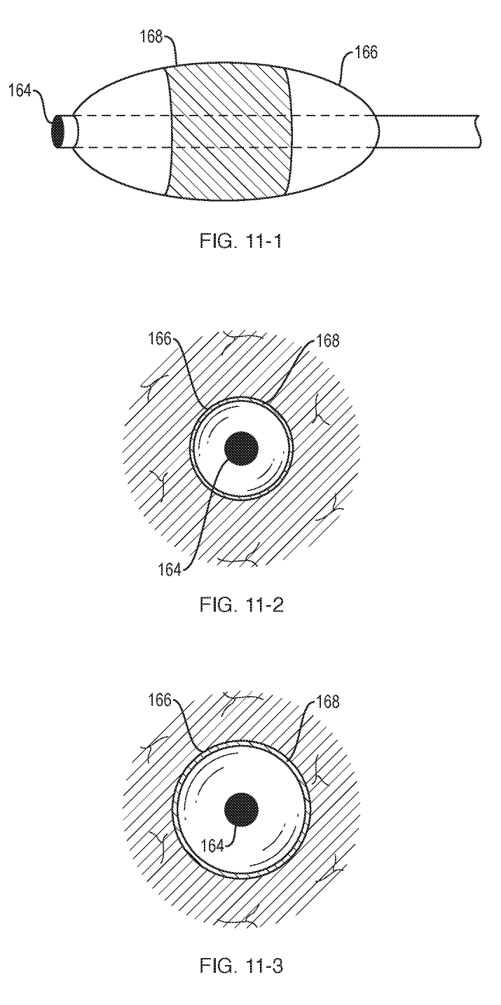

As shown in FIGS. 10 and 11-1, the first electrode 164 of the fistula creating and expanding device 160 is activated/charged/powered upon while or just before making contact with the tissue wall located within a central area of the deployed tissue fastening device 100. The heat generated by the first electrode 164 causes cutting/ablation of the tissue wall. While electrode activation and tissue cutting are occurring, an operator advances the fistula creating and expanding device 160 through the deployed tissue fastening device 100 until of the tissue walls have been pierced. Once the fistula creating and expanding device 160 passes through both tissue walls compressed by the deployed tissue fastening device 100, the power to the first electrode 164 is turned off. The second electrode(s) 168 is now in contact with the edges of the tissue walls that have just been cut by the first electrode 164. While the operator inflates the balloon 166, the operator simultaneously activates the second electrode 168. The heat from the second electrode 168 continually cuts/ablates the edges of the fistula while the balloon 166 expands, thus increasing the size of the fistula—see FIGS. 11-2 and 11-3. After the balloon 168 has reached a desired diameter, the second electrode 168 is deactivated, the balloon 166 is deflated and the distal end of the fistula creating and expanding device 160 is retracted from within the deployed tissue fastening device 100. The first and second electrodes 164 and 168 individually operate in a monopolar mode in conjunction with the patient pad 82.

The fistula creating and expanding device 160 may be guided and deployed from a device such as that shown in FIG. 1. The handle of the fistula creating and expanding device 160 also includes a first switch for activating the first electrode 164 and a second switch for activating the second electrode 168. In one embodiment, the second switch is deactivated when the first electrode 164 is activated and the first switch is deactivated when the second electrode 168 is activated. In another embodiment, the activation features for the first and second electrodes 164, 168 are included in a single multi-throw switch.

### Embodiments

A. A system comprising: a tissue fastening tool for applying pressure to a periphery around an area of a first biological tissue and a second biological tissue of a body; a power source; and a fistula creating device comprising: a handle portion configured to be used exterior to a body; a catheter comprising a proximal end coupled to the handle portion and a distal end; and one or more electrodes located at the distal end of the catheter, wherein the one or more electrodes are in electrical communication with the power source and are configured to cut the first and second biological tissue within the area when charged by the power source.

B. The system of A, wherein the one or more electrodes comprises two electrodes configured to operate in a bipolar manner.

C. The system of B, wherein a first electrode of the two electrodes is located on an exterior surface of the catheter, wherein a second electrode of the two electrodes is slidably received within the catheter, wherein at least one of the first or second electrodes comprises insulation for isolating the first electrode from the second electrode.

D. The system of B, wherein a first electrode of the two electrodes is located on a first side of a lumen of the catheter, wherein a second electrode of the two electrodes is located on a second side of the catheter, further comprising at least one insulator located between the first electrode and the second electrode.

E. The system of D, wherein the first and second electrodes are slidably received within the catheter.

F. The system of A, further comprising a patient pad in electrical communication with the power source, wherein the one or more electrodes are powered by the power source in a monopolar manner in conjunction with the patient pad.

G. The system of A, further comprising: a balloon attached to the catheter proximal from the distal end of the catheter; and one or more balloon electrodes attached to an exterior surface of the balloon, wherein the one or more electrodes comprises a tip electrode located at a distal end of the catheter, wherein the catheter comprises a lumen exposed at a distal end to an interior of the balloon and at a proximal end to the handle portion, wherein the lumen is configured to receive at least one of a gas or a fluid from the handle portion and pass the received gas or fluid to the balloon in order to inflate the balloon.

H. A method comprising: applying pressure to a periphery around an area of a first biological tissue and a second biological tissue of a body; applying power to one or more electrodes; and cutting the first and second biological tissues while advancing the powered one or more electrodes to and through the area, thereby creating a fistula between the first and second biological tissues.

I. The method of H, further comprising deactivating the one or more electrodes once the powered one or more electrodes pass through the first and second biological tissues.

J. The method of H, further comprising: while inflating a balloon located proximal from the distal end of the catheter, activating one or more balloon electrodes attached to an exterior surface of the balloon.

K. The method of J, wherein the catheter comprises a lumen exposed at a distal end to an interior of the balloon and a proximal end to a handle portion, wherein inflating comprises delivering at least one of a gas or a fluid from the handle portion to the balloon via the lumen in order to inflate the balloon.

L. The method of K, wherein the one or more electrodes comprises a tip electrode located at a distal end of the catheter, wherein applying power to the one or more electrodes comprises applying power to the tip electrode in association with a patient pad located external to the body.

M. The method of L, wherein applying power to the tip electrode is performed before inflating the balloon.

The description of the invention is merely exemplary in nature and variations that do not depart from the gist of the invention are intended to be within the scope of the invention. Such variations are not to be regarded as a departure from the spirit and scope of the invention.

Although the preferable embodiments of the present invention have been described hitherto, the present invention is not limited to these embodiments. Additions, omissions, substitutions, and other modifications can be made without departing from the spirit or scope of the present invention.

In addition, the invention is not limited by the above description and is limited by only the scope of appended claims.

What is claimed is:

1. A system comprising:
a tissue fastening tool for applying pressure to a periphery around an area of a first biological tissue and a second biological tissue of a body;
a power source; and
a fistula creating device comprising:
a handle portion configured to be used exterior to a body;
a catheter comprising a proximal end coupled to the handle portion and a distal end; and
one or more electrodes located at the distal end of the catheter, wherein the one or more electrodes are in electrical communication with the power source and are configured to cut the first and second biological tissue within the area when charged by the power source.

2. The system of claim 1, wherein the one or more electrodes comprises two electrodes configured to operate in a bipolar manner.

3. The system of claim 2, wherein a first electrode of the two electrodes is located on an exterior surface of the catheter, wherein a second electrode of the two electrodes is slidably received within the catheter, wherein at least one of the first or second electrodes comprises insulation for isolating the first electrode from the second electrode.

4. The system of claim 2, wherein a first electrode of the two electrodes is located on a first side of a lumen of the catheter, wherein a second electrode of the two electrodes is located on a second side of the catheter, further comprising at least one insulator located between the first electrode and the second electrode.

5. The system of claim 4, wherein the first and second electrodes are slidably received within the catheter.

6. The system of claim 1, further comprising a patient pad in electrical communication with the power source, wherein the one or more electrodes are powered by the power source in a monopolar manner in conjunction with the patient pad.

7. The system of claim 1, further comprising:
a balloon attached to the catheter proximal from the distal end of the catheter; and
one or more balloon electrodes attached to an exterior surface of the balloon,
wherein the one or more electrodes comprises a tip electrode located at a distal end of the catheter,
wherein the catheter comprises a lumen exposed at a distal end to an interior of the balloon and at a proximal end to the handle portion, wherein the lumen is configured to receive at least one of a gas or a fluid from the handle portion and pass the received gas or fluid to the balloon in order to inflate the balloon.

8. A method comprising:
applying pressure to a periphery around an area of a first biological tissue and a second biological tissue of a body;
applying power to one or more electrodes; and
cutting the first and second biological tissues while advancing the powered one or more electrodes to and through the area, thereby creating a fistula between the first and second biological tissues.

9. The method of claim 8, further comprising deactivating the one or more electrodes once the powered one or more electrodes pass through the first and second biological tissues.

10. The method of claim 8, further comprising:
while inflating a balloon located proximal from the distal end of the catheter, activating one or more balloon electrodes attached to an exterior surface of the balloon.

11. The method of claim 10, wherein the catheter comprises a lumen exposed at a distal end to an interior of the balloon and a proximal end to a handle portion,
wherein inflating comprises delivering at least one of a gas or a fluid from the handle portion to the balloon via the lumen in order to inflate the balloon.

12. The method of claim 11, wherein the one or more electrodes comprises a tip electrode located at a distal end of the catheter, wherein applying power to the one or more electrodes comprises applying power to the tip electrode in association with a patient pad located external to the body.

13. The method of claim 12, wherein applying power to the tip electrode is performed before inflating the balloon.
